(19) United States
(12) Patent Application Publication
(10) Pub. No.: US 2005/0222601 A1
(43) Pub. Date: Oct. 6, 2005

(54) Laparoscopic instrument

(76) Inventor: Martin Erhard, Silbertal (AT)

Correspondence Address: WENDEROTH, LIND & PONACK, L.L.P., 2033 K STREET N. W., SUITE 800, WASHINGTON, DC, 20006-1021, US

(21) Appl. No.: 11/098,436

(22) Filed: Apr. 5, 2005

(30) Foreign Application Priority Data
Apr. 6, 2004 (AT) A 602/2004

(51) Int. Cl. A61B 17/00

(52) U.S. Cl. 606/190

(57) ABSTRACT

A laparoscopic instrument comprises a handle (1), a shaft (2) supported by the handle (1), and a working tip (3) disposed at the distal end of the shaft (2), the shaft (2) comprising a distal curvable section (4) and, for the adjustment of the curvature of the curvable section (4), a tension element (7) formed by a wire or cord extends from the proximity of the handle (1) across the curvable section (4) and is secured at the distal side of the curvable section (4) on the shaft (2) or on the working tip (3), and an actuation member (5), disposed in the proximity of the handle (1), cooperates with the tension element (7) for exerting a tensile action onto the tension element (7), and wherein over the curvable section (4) several guide brackets (8) for the tension element (7) are disposed spaced apart from one another in the longitudinal direction of the curvable section (4). Over the curvable section (4) extends furthermore a leaf spring (19) which, in the untensioned state, is straight, wherein the raising of the tension element (7) from the leaf spring (19) is limited by the guide brackets (8).

### BACKGROUND OF THE INVENTION

a) Field of the Invention

The invention relates to a laparoscopic instrument with a handle, a shaft supported by the handle, and a working tip disposed at the distal end of the shaft, the shaft comprising a distal curvable section. To adjust the curvature of the curvable section, a tension element formed of a wire or a cord extends from the proximity of the handle across the curvable section and is secured on the distal side of the curvable section on the shaft or on the working tip. An actuation member disposed in the proximity of the handle cooperates with the tension element for the tensile action on the tension element and, over the curvable section, several guide brackets for the tension element are disposed spaced apart from one another in the longitudinal direction.

b) Description of Related Prior Art

Laparoscopic instruments are surgical instruments for laparoscopic operations. In such, an access opening into a body cavity is created at the site selected for the intervention. Trocars are conventionally employed to create such an access opening. Such trocars are implemented in the form of a shell and have a passage channel, through which by means of a laparoscopic instruments the operation is performed. This surgical technique is among the minimally invasive surgical techniques.

Known is a laparoscopic instrument with a handle, a shaft supported by the handle, and a working tip disposed at the distal end of the shaft, in which a distal curvable section of the shaft is formed of several links articulated with one another. Next to a tension element, which can be acted upon by an actuation member disposed on the handle, extends a second tension line through the curvable section, which is secured in place, on the one hand, on the handle, on the other hand, on the working tip, and in which a helical spring is disposed. This second cord line serves for straightening the curvable section when the tension element cooperating with the actuation part is not tensioned and displaced via this actuation part, but is relaxed. The adjustable curvature of the curvable section serves for moving the working tip around structures, for example hollow organs or vessels, in the intracorporal region. For example, with the working tip subsequently an implant, a suture or another object can be pulled around a hollow organ or a vessel. Of disadvantage in this conventional instrument is inter alia its relatively complex structure, which results in corresponding costs for this instrument. Furthermore, to clean this instrument after an operation has been performed without residues remaining is virtually impossible. Between the links of the curvable section and on the tension cords blood and body fluids collect during the operation, residues of which remain even after rinsing and sterilizing the instrument. These residues entail the risk of infection.

Known is furthermore, for example from DE 41 33 800 C1, a laparoscopic instrument, in which, specifically by means of an actuation member disposed on the handle, a leaf spring can be slid from the distal end of a tube attached on a handle. In the state in which it is disposed within the tube, the leaf spring is tensioned and, upon being slid out of the tube, assumes its specified curvature, which it has in the absence of external forces acting on it. The distal end of this leaf spring forms here the working tip of the instrument. This working tip serves, for example, for dissecting tissue and can be provided with a slit, in which an object, for example a suture, can be suspended in order to pull it around a hollow organ or a vessel. Of disadvantage in this instrument is inter alia its problematic cleaning, in particular of body fluids penetrated into the front end of the tube. There are also restrictions with respect to changes of the radius of curvature in the section adjoining the working tip.

DE 43 25 969 C1 discloses a circumnavigation instrument with a longish arm which can be placed about an object and which can be extended and bent off, at whose free end is disposed a holding tool, the arm being implemented, at least over a portion of its length, as a hose of an extensible material. The longitudinal extensibility of the hose is different at its two opposing sides and the interior of the hose is connected to a pressure source filling a fluid medium under pressure into the hose.

A laparoscopic instrument of the above described type is disclosed in U.S. Pat. No. 6,645,218 B1. The curvable section of the shaft is elastic, a row of wedge-shaped slots extending from the top side of the shaft and a row of wedge-shaped slots each disposed between the slots, extending from the bottom side. Starting from an actuation member disposed on a handle are two parallel tension elements, extending across the shaft up to a securement site located distally to the curvable section. In the curvable section these tension elements are guided by the sections of the shaft spaced apart by the wedge-shaped slots, which sections consequently can be viewed as guide brackets for the tension elements. In its curvable section the lateral rigidity of the shaft of the instruments is relatively low.

### AIM AND SUMMARY OF THE INVENTION

The aim of the invention is to provide an improved laparoscopic instrument of the above described type. According to the invention this is attained through a laparoscopic instrument with a handle, a shaft supported by a handle, and a working tip disposed at the distal end of the shaft, the shaft comprising a distal curvable section. For the adjustment of the curvature of the curvable section, a tension element, formed by a wire or cord, extends from the proximity of the handle across the curvable section and is secured on the distal side of the curvable section on the shaft or on the working tip, and an actuation member, disposed in the proximity of the handle, cooperates with the tension element for exerting tensile action onto the tension element. Across the curvable section, furthermore, a leaf spring extends, which, in the untensioned state, is straight, and over the curvable section are disposed several guide brackets, spaced apart from one another in the longitudinal direction of the curvable section, opposite the leaf spring to limit the raising of the tension element.

Through the implementation according to the invention, in which a leaf spring extends over the curvable section, high flexural strength is attained in the lateral direction, i.e. in the direction perpendicular to the plane in which the shaft lies in its curved state. Thereby an extremely good force transmission in this lateral direction can be attained, whereby, for example, performing blunt dissections is facilitated.

Through the invention furthermore a highly cost-effective fabrication of the instrument is made possible. The instrument can thereby inter alia also be implemented as a disposable instrument for single-use, such that cleaning problems no longer apply and the risk of infection is reduced.

An instrument according to the invention can be implemented furthermore in such a way that the residue-free cleaning becomes possible. The curvable section is herein advantageously encompassed by an outer cover hose, which preferably extends over the entire length of the shaft.

In a preferred embodiment of the invention a formed part of a synthetic material is provided, which extends over the curvable section and which comprises the guide brackets, each of the successive guide brackets being connected by connecting sections of the formed part, which are connected with the free ends of the shanks of the U-shaped guide brackets. The leaf spring extends here between the shanks of the guide brackets.

The curvable section is preferably filled and cast with silicon to form a smooth outer contour over the length of the curvable section, the development of an outer contour in the form of a cylindrical shell being especially preferred. Thereby, in particular together with the preferably employed outer cover hose, it is possible to attain a substantially smooth outer surface at least in the straightened state of the curvable section.

Further advantages and details of the invention will be explained in the following in conjunction with the embodiment example depicted in the drawing, based on which further aims of the invention are evident.

### BRIEF DESCRIPTION OF THE DRAWING

In the drawing depict:

FIG. 1 a longitudinal section through an embodiment example of a laparoscopic instrument according to the invention,

FIG. 2 an enlarged cutout from FIG. 1,

FIG. 3 a cross section along line A-A of FIG. 2,

FIG. 4 a front portion of the shaft (without cover hose and silicon casting compound) in perspective representation,

FIG. 5 a perspective representation of the formed part,

FIGS. 6, 7 and 8 a view from below, a side view and a view from above of the formed part.

### DESCRIPTION OF THE PREFERRED EMBODIMENT EXAMPLES

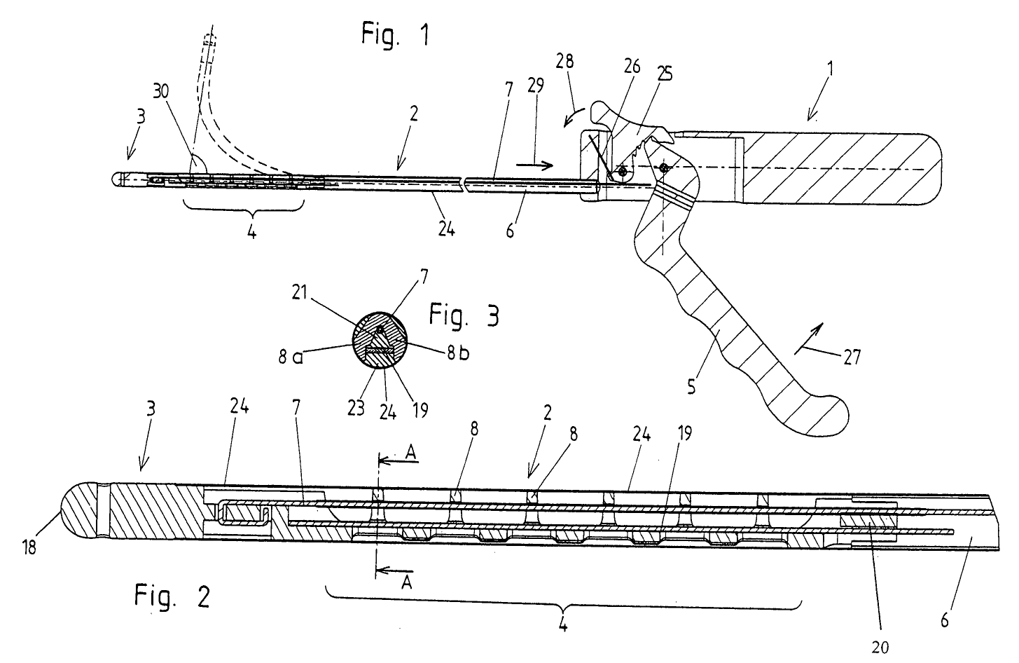

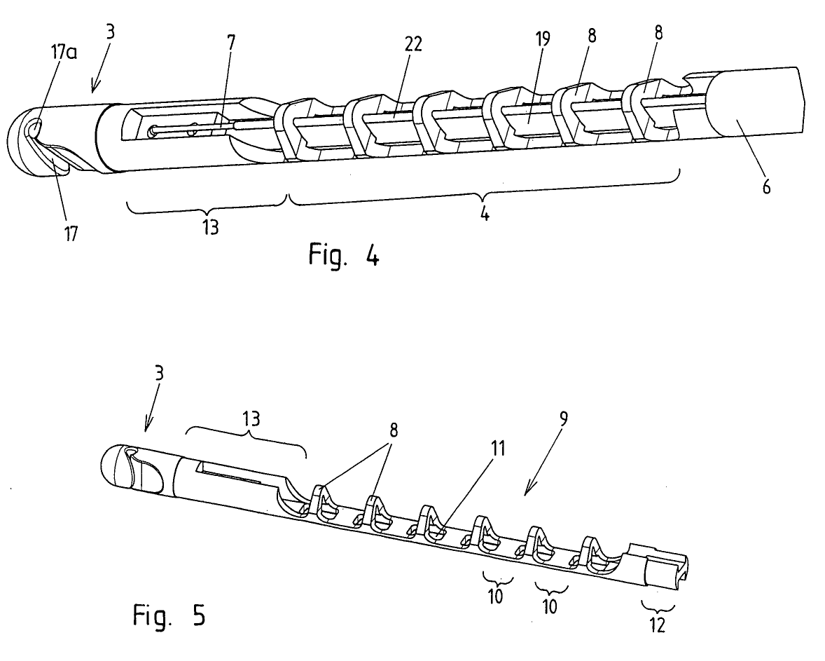

An embodiment example of a laparoscopic instrument according to the invention is depicted in the Figures. The instrument comprises a handle 1, a shaft 2 secured on the handle, and a working tip 3 disposed at the distal end of the shaft 2. The shaft 2 comprises a distal curvable section 4, which can be adjusted between a straight position, depicted in FIG. 1 by continuous lines, and a curved position, indicated in FIG. 1 by dot-dash lines. The curvable section 4 is located distal to the longitudinal center of the shaft 2, preferably in a region adjoining the working tip 3, the distance of the curvable section 4 from the working tip 3 favorably amounting to less than one half of the length of the curvable section 4. To adjust the curvature of the curvable section 4 serves an actuation member 5 located in the proximity of the handle 1, which actuation member in the depicted embodiment example is formed by an actuation lever swivelably pivoted on handle 1.

The proximal portion of shaft 2 is formed by a tube 6, whose proximal end section projects into a bore in handle 1 and is secured, for example adhered, in it. The curvable section 4 is secured, for example adhered, on the distal end of the tube 6.

To adjust the curvature of the curvable section 4 serves furthermore a tension element 7, which is formed by a wire or a cord (comprised of several strands). The tension element 7 extends from the proximity of the handle 1 across the curvable section 4 and on the distal side of the curvable section is secured on the shaft 2. On the end located on the side of the handle 1 the tension element 7 is secured on the actuation member 5.

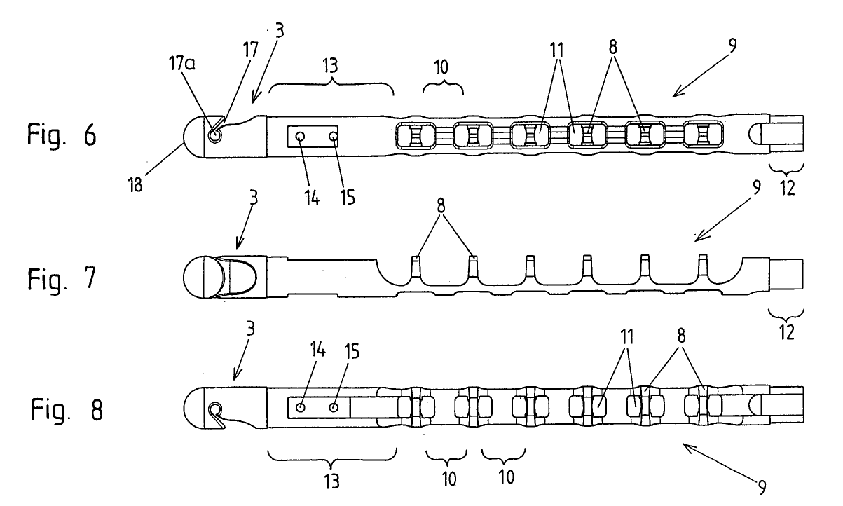

Over the curvable section 4 several guide brackets 8 are disposed in the longitudinal direction spaced apart from one another. In the depicted embodiment example, these guide brackets 8 are portions of a formed part 9 (cf. FIG. 5 to 8). The successive guide brackets 8 are each connected with one another by a connecting section 10 of the formed part 9. In cross section of shaft 2 (cf. FIG. 3), the guide brackets 8 have the shape of a U and comprise two shanks 8a, 8b, which are connected with one another via a bracket section. The connecting sections 10 are formed on at the ends of shanks 8a, 8b remote from the bracket section. In the proximity of each guide bracket 8 an opening 111 is left vacant in the connecting sections 10, whereby the flexibility of the formed part 9 is increased.

The formed part 9 comprises at its proximal end an insertion section 12 to insert and adhere it in the tube 6. Distally adjoining the section (which coincides with the curvable section 4 of the shaft 2), formed by the guide brackets 8 and connecting sections 10, is a securement section 13, in which the tension element 7 is secured on shaft 2. The securement section 13 is developed with sufficient stability for this purpose, and for securing the tension element 7 serve the two through-openings 14, 15, through which the tension element 7 is guided and bent over and subsequently can be adhered.

Distally adjoining the securement section 13 is the working tip 3, which in this embodiment example is consequently formed integrally with the formed part 9. The working tip 3 represents the section of the formed part 9 projecting beyond the outer cover hose 24 (which will be described later). The working tip 3 is provided with a slot 17 starting from one side of the working tip 3, which slot initially tapers down and, adjoining the taper, has an enlarged region 17a. The front side or distal end 18 of the working tip 3 is rounded off. With the working tip 3 a blunt dissection can be carried out.

Across the curvable section 4 extends furthermore a leaf spring 19, which, in the untensioned state (i.e. in the absence of external forces acting on it) extends in a straight course in the longitudinal direction. Through the flat leaf-form shape, high flexural strength is attained in the lateral direction, i.e. in the direction perpendicular to the plane in which shaft 2 lies in its curved state. This lateral flexural strength is maintained in the curved as well as also in the straightened or extended state of shaft 2. Thereby an extremely good force transmission in this lateral direction can be attained, whereby, for example, blunt dissecting is facilitated.

The flexural strength in the direction of the curvature (thus in the direction upward starting from the extended state in FIG. 1) depends on the elastic force of the leaf spring 19. Since through the actuation member 5 an extremely high force transmission can be attained, the leaf spring 19 can be laid out correspondingly rigidly, such that a relatively high flexural strength is also attained in this direction. In the opposite direction (downward in FIG. 1) high flexural strength results through the holding force of the tension element 7.

The leaf spring 19 is disposed nondisplaceably in the axial direction of the shaft.

In the depicted embodiment example the leaf spring 19 extends between the shanks 8a, 8b of the guide brackets, and in the region between two guide brackets 8 it extends on the side of the connecting section 10 facing the guide brackets 8. The width of the leaf spring 19 corresponds to the distance between shanks 8a, 8b and the leaf spring 19 is secured against being raised from the connecting sections 10 by steps of the shanks 8a, 8b with narrow tolerance.

In the distal end region the leaf spring 19 is adhered on the securement section 13. The proximal end section extends underneath a web 20 of the insertion section 12 of formed part 9 and proximally projects minimally beyond the formed part 9.

Together with the leaf spring 19, a particular guide bracket 8 delimits a channel 21, through which extends the tension element 7.

The tension element 7 is encompassed over the length of the curvable section 4 by a flexible slip tube 22, which is preferably formed by a Teflon tube, in order to reduce the kinetic friction of the tension element 7.

After the leaf spring 19 has been set into the formed part 9 and has been adhered to it, and the tension element 7 has been secured in the through-openings 14, 15 and, with the slip tube 22 slid on, extends through the channels 21, these parts are filled in with a casting compound, preferably silicon, the parts being placed into a corresponding mould. Thereby a part is formed with a smooth outer contour between the working tip 3 and the insertion section 12, which preferably has the form of a cylindrical shell. This part comprises the curvable section 4 and extends over the length of the formed part 9 with the exception of the working tip 3 and of the insertion section 12. For the sake of clarity, the casting compound 23 is only shown in FIG. 3.

The cast part is subsequently adhered into the tube 6. Subsequently a shrink hose is placed over the length of the shaft 2, which is shrunk thereon in the following. Thereby an external cover hose 24 results having the form evident in FIG. 1 to 3. This cover hose 24 has at least in the straightened state of the curvable section 4 a substantially smooth outer surface over the entire length of shaft 2 and closes off the shaft 2 against the penetration of fluids. By developing this shaft 2 to be hermetically closed, the residue-free cleaning of the instrument after an operation is made possible. Through the substantially smooth implementation of the outer surface of the cover hose 24 this cleaning is still further facilitated.

To secure in place a set swivel position of the actuation member 5, and consequently a set curvature of the curvable section 4, a latching device is available which cooperates with the actuation member 5. This latching device comprises a swivelable latching part 25, which is provided with a toothing cooperating with the actuation member 5. Through a spring 26 the toothing is pressed against a latching element of the actuation member 5. Upon swiveling the actuation member 5 in the direction of arrow 27, the latching element latches into the corresponding tooth of the toothing of latching part 25. The latching can be detached again through the manual swiveling of latching part 25 in the direction of arrow 28.

During the swiveling of actuation member 5 in the direction of arrow 27, the tension element 7 is displaced in the direction of arrow 29, and the curvable section 4 is increasingly curved by overcoming the reset force of leaf spring 19. The curvature of the curvable section 4 is herein continuously adjustable between the straight position and the maximally curved position. In an instrument according to the invention in the maximally curved position an angle 30 of the swiveling of the working tip 3 by more than 90° is attained, and in practice angles of more than 110° can be attained.

In the curved state of the curvable section a force in the direction of raising the tension element from the leaf spring 19 acts onto the tension element. Through the guide brackets 8 such a raising of the tension element 7 with respect to the leaf spring 19 is limited. In the embodiment example depicted, the tension element has substantially the same distance from the leaf spring 19 in the straight and in the curved state of the curvable section. The formed part 9 is comprised of a synthetic material, for example of polysulfone, which, on the one hand, has sufficient flexibility to be bent without suffering damage, and, on the other hand, has sufficient strength in order to be able to withstand the forces exerted by the tension element 7. The cover hose 24 can be formed for example of a PTFE shrink hose. Instead of Teflon, another soft synthetic material approved for medical application can be employed. The leaf spring 19 can for example be comprised of special steel. The tension element 7 can be formed for example by a metal wire or metal cord.

With an instrument according to the invention blunt dissections, in which tissue layers are separated from one another, can advantageously be performed. With the working tip 3 herein structures in the intracorporal region, for example hollow organs or vessels, can be circumnavigated, for which purpose the curvature or flexure of the curvable section 4 is correspondingly set. With the working tip 3, furthermore, objects, for example sutures or implants, can be drawn about hollow organs or vessels. An instrument according to the invention can be advantageously employed for example in the transplantation of a stomach band.

Distinctive modifications of the described embodiment example of the invention are conceivable and possible without going beyond the scope of the invention. It would be conceivable and possible, for example, to secure in place the guide brackets directly on the leaf spring 19, and in this case a formed part extending over the length of the curvable section 4 could also be omitted. The guide brackets 8 could for example be welded with the two ends of their shanks onto the leaf spring 19. The guide brackets 8 could here be formed for example by a bent metal wire.

It would furthermore also be conceivable and possible to omit the casting compound of silicon if a cover hose is employed comprised of a sufficiently stable material, in order to avoid the formation of unevennesses that are too pronounced on the outside of the cover hose, which impair the surgical use of the instrument. The cover hose could also be developed in a manner other than by a shrink hose. It would also be conceivable and possible to omit the cover hose and to provide only a casting compound, which forms the outer surface of the shaft in the curvable section, and this would be possible in particular if the instrument is only employed as a disposable instrument for single use.

The leaf spring 19 could, for example, also be comprised of a shape-storing alloy (a material with so-called shape memory). The reset properties of such shape-storing alloys are also denoted as “pseudo-elastic”. Here, for example nickel-titanium alloys could be employed.

Actuation members other than swivelable handles are also conceivable and possible. A rotatably supported nut could, for example, also be provided, into the threads of which a threaded rod projects secured against twisting, on which the tension element 7 is secured. In principle, more than a single tension element could also be provided. The guide brackets 8 could also be developed such that they are closed in the region between tension element 7 and leaf spring 19, whereby separate channels could be developed for the through-guidance of tension element 7 and leaf spring 19.

As is evident based on the preceding description, the scope of the invention is not limited to the depicted embodiment examples, but should be determined with reference to the attached claims together with their full range of feasible equivalents. While the preceding description and the drawing represent the invention, it is obvious to a person of skill in the art that distinctive modifications can be carried out therein without leaving the true essence and scope of the invention.

### LEGEND TO THE REFERENCE NUMBERS

1 Handle
2 Shaft
3 Working tip
4 Curvable section
5 Actuation member
6 Tube
7 Tension element
8 Guide brackets
8a Shank
8b Shank
9 Formed part
10 Connecting section
11 Opening
12 Insertion section
13 Securement section
14 Through-opening
15 Through-opening
17 Slit
17a Enlarged region
18 Distal end
19 Spring element
20 Web
21 Channel
22 Slip tube
23 Casting compound
24 Cover hose
25 Latching part
26 Spring
27 Arrow
28 Arrow
29 Arrow
30 Angle

1. Laparoscopic instrument with a handle (1), a shaft (2) supported by the handle (1), and a working tip (3) disposed at the distal end of the shaft (2), the shaft (2) comprising a distal curvable section (4) and, for the adjustment of the curvature of the curvable section (4), a tension element (7) formed by a wire or cord extends from the proximity of the handle (1) across the curvable section (4) and is secured at the distal side of the curvable section (4) on the shaft (2) or on the working tip (3), and an actuation member (5) disposed in the proximity of the handle (1) cooperates with the tension element (7) for exerting tensile action onto the tension element (7), and wherein over the curvable section (4), furthermore, a leaf spring (19) extends which, in the untensioned state, is straight, and over the curvable section (4) several guide brackets (8) are disposed spaced apart in the longitudinal direction of the curvable section (4) for limiting the raising of the tension element (7) from the leaf spring (19).

2. Laparoscopic instrument as claimed in claim 1, wherein a particular guide bracket (8) together with the spring element (19) delimit a channel (21), through which extends the tension element (7).

3. Laparoscopic instrument as claimed in claim 1, wherein a particular guide bracket (8) is implemented such that it is U-shaped in cross section of the shaft (2).

4. Laparoscopic instrument as claimed in claim 1, wherein a formed part (9) of a synthetic material is provided, which extends over the curvable section (4) and comprises the guide brackets (8).

5. Laparoscopic instrument as claimed in claim 4, wherein successive guide brackets (8) are each connected by a connecting section (10) of the formed part (9).

6. Laparoscopic instrument as claimed in claim 5, wherein the connecting sections (10) are formed on at the ends of shanks (8a, 8b) of the U-shaped guide brackets (8).

7. Laparoscopic instrument as claimed in claim 4, wherein the formed part (9) also forms the working tip (3).

8. Laparoscopic instrument as claimed in claim 4, wherein the leaf spring (19) is secured on the formed part (9) distally and/or proximally to the curvable section (4).

9. Laparoscopic instrument as claimed in claim 8, wherein the leaf spring is adhered on the formed part.

10. Laparoscopic instrument as claimed in claim 4, wherein the formed part (9) is secured on the distal end of a tube (6), which is secured with its proximal end on the handle (1).

11. Laparoscopic instrument as claimed in claim 1, wherein at least over the length of the curvable section (4) the tension element (7) is encompassed by a flexible slip tube (22).

12. Laparoscopic instrument as claimed in claim 1, wherein at least the curvable section (4) is filled with a casting compound (23) to form a smooth outer contour at least over the length of the curvable section (4).

13. Laparoscopic instrument as claimed in claim 12, wherein the casting compound is silicon.

14. Laparoscopic instrument as claimed in claim 12, wherein the curvable section is cast to form an outer contour in the shape of a cylindrical shell.

15. Laparoscopic instrument as claimed in claim 1, wherein the curvable section (4) is encompassed by an outer cover hose (24).

16. Laparoscopic instrument as claimed in claim 15, wherein the outer cover hose (24) extends over the entire length of the shaft (2).

17. Laparoscopic instrument as claimed in claim 1, wherein the actuation member (5) is formed by an actuation grip, swivelably pivoted on the handle (1), on which the tension element (7) is secured.

18. Laparoscopic instrument as claimed in claim 17, wherein a detachable latching device with a latching part (15) cooperating with the actuation member (5) is provided for securing in place a set swivel position of the actuation member (5).

19. Laparoscopic instrument as claimed in claim 1, wherein the working tip (3) in the position of greatest curvature of the curvable section (4) is swiveled by an angle (30) of more than 90° with respect to the straight starting position of the curvable section (4).

20. Laparoscopic instrument as claimed in claim 2, wherein a particular guide bracket (8) is implemented such that it is U-shaped in cross section of the shaft (2).
